(19) United States
(12) Patent Application Publication
(10) Pub. No.: US 2026/0094723 A1
(43) Pub. Date: Apr. 2, 2026

(54) System and Method of Brain Age Identification for Predicting Neuro-Degenerative Disease

(71) Applicant: Vanderbilt University, Nashville, TN (US)

(72) Inventors: Chenyu Gao, Nashville, TN (US); Bennett A. Landman, Nashville, TN (US); Michael E. Kim, Nashville, TN (US)

(21) Appl. No.: 19/345,706

(22) Filed: Sep. 30, 2025

Related U.S. Application Data
(60) Provisional application No. 63/701,861, filed on Oct. 1, 2024.

(51) Int. Cl. G16H 50/30; G16H 30/40

(52) U.S. Cl. CPC G16H 50/30; G16H 30/40

(57) ABSTRACT

Calculating a brain age predicts progression from cognitively normal status to cognitive impairment. Software acquires diffusion magnetic resonance images (dMRI) and T1 weighted magnetic resonance images (T1w MRI) of the brain and calculate a pair of diffusion tensor imaging (DTI) scalar maps. The scalar maps may be a fractional anisotropy (FA) scalar map and/or a mean diffusivity (MD) scalar map. Concatenating the FA scalar map and the MD scalar map form a frame of scalar map data. The method includes a rigid transformation from scalar map data to the T1w images to store a rigid transformed image; applying an affine transformation from the rigid transformed image to a brain template; storing a rigid plus affine transformed image; and applying a non-rigid transformation to warp the rigid plus affine transformed image to the selected brain template to form a non-rigid white matter age input to a brain age calculation model.

### CROSS REFERENCE TO RELATED APPLICATIONS

This application claims priority to and incorporates by reference U.S. Provisional Patent Application Ser. No. 63/701,861 filed on Oct. 1, 2024.

### STATEMENT OF GOVERNMENT INTEREST

This invention was made with government support under NIH Grants 1R01EB017230, U24AG074855, R01MH121620, K01EB032898, and K01-AG073584. This invention was also made with government support under National Center for Advancing Translational Sciences (NCATS) Clinical Translational Science Award (CTSA) Program, Award Number 5UL1TR002243-03. The government has certain rights in the invention.

### SUMMARY

In one embodiment, a computer implemented method of preparing images of a subject's brain is used for calculating a brain age for the subject. The method uses a computer having a processor and computer readable memory storing software to implement steps that include acquiring diffusion magnetic resonance images (dMRI) and T1 weighted magnetic resonance images (T1w MRI) of the brain of the subject; calculating a pair of diffusion tensor imaging (DTI) scalar maps having a fractional anisotropy (FA) scalar map and mean diffusivity (MD) scalar map; concatenating the FA scalar map and the MD scalar map to form a frame of scalar map data; applying a rigid transformation from the frames of scalar map data to the T1w images to store a rigid transformed image in the computer memory; applying an affine transformation from the rigid transformed image to a selected brain template and storing a rigid plus affine transformed image in the computer memory; and applying a non-rigid transformation to warp the rigid plus affine transformed image to the selected brain template to form a non-rigid white matter age input to a brain age calculation model.

### BRIEF DESCRIPTION OF THE FIGURES

FIG. 1. Brain age estimation frameworks have proven effective for using affinely aligned brain images to identify common patterns of aging, with deviations from these patterns being related to disease. A common theme of existing brain age estimation methods has been using T1w MRI, denoted as “GM age” in the first row. Among them, there have been many innovations in network design, such as DeepBrainNet (DBN) [7] and the 3D convolutional neural network of TSAN [13]. T1w MRI lacks detail in white matter (WM). Here, this disclosure takes the two most commonly used modalities for characterizing WM microstructure, fractional anisotropy (FA), and mean diffusivity (MD), and it evaluates brain age estimation in two contexts. First, the systems and methods herein examine the direct substitution of FA and MD for T1w image, which this disclosure denoted as “WM age affine” in the second row. A substantial amount of macrostructural differences is still present in WM age affine, notably ventricle enlargement. To isolate the microstructural changes, this disclosure applies non-rigid (deformable) registration into template space to mitigate the macrostructural changes and produce the “WM age nonrigid” in the third row. This disclosure explored the relative timing of changes in these brain age variants and their relative explainability in the context of mild cognitive impairment.

FIG. 2. The fractional anisotropy (FA) and mean diffusivity (MD) images are calculated from volumes with b-value≤1500 s/mm² extracted from preprocessed diffusion MRI data. Rigid registration (the green line) between b0 image and T1w image, and affine and non-rigid (deformable) registrations (the purple line) between T1w image and MNI152 T1w template are performed and concatenated to form the transformation from b0 space to MNI152 space. A brain mask is computed from T1w image with SLANT whole brain segmentation pipeline and applied to the FA and MD images. Typically, a diffusion-weighted MR image contains a series of 3D images (of the same brain). Each of the 3D images was acquired with different magnetic gradients (with varying directions and strengths, where this disclosure used b-value to denote the strength). And typically, the first (few) 3D image(s) was acquired with minimally weighted gradient field, and therefore are referred to as b0 image.

FIG. 3. As neurodegeneration progresses, estimated brain age generally deviates more from chronological age, as reflected by the shape of the density distribution of the brain age gap (estimated brain age−chronological age) and the mean of the absolute value of the BAG value (denoted herein as |BAG|*). CN* are participants cognitively normal at present but diagnosed with MCI in follow-up sessions. Scatters beyond the age range (45 to 90 years) used for training are colored gray and excluded from calculation of |BAG|* and from subsequent analysis.

FIG. 4. Data points from four diagnosis groups are matched regarding age and sex (and time to last CN and time to first MCI for matching CN and CN* data points). The differences between WM age nonrigid and GM age (ours) are adjusted by the mean of the differences for the CN group. Wilcox on signed-rank tests show significant difference between WM age nonrigid and GM age (ours) on both CN* and AD participants.

FIG. 5A. The longitudinal data from CN* participants are used for MCI prediction from n years pre-diagnosis in two experimental setups. In the “Global Model” setup (left subplot), WM age nonrigid shows an advantage from 0 to approximately 3.5 years before MCI. In the “Time-Specific Models” setup (right subplot), WM age nonrigid shows an advantage up to approximately 4-5 years before MCI. However, these advantages are not statistically significant, as indicated by the overlapping confidence intervals. The global model and the time-specific models both show that the WM age-derived features—or their combination with other features—outperform other features, in predicting whether a CN participant will transition to MCI, from 0 to 4 years before diagnosis. The vertical lines in gray color mark out the mean age of each subset. The sample size of each subset is overlaid on the violin plot. At T−5 (5 years before MCI diagnosis), features derived from GM age (DBN) outperformed other features when using random forest classifiers under the global model setup, achieving an AUC of 0.78; logistic regression and linear SVM classifiers using features derived from GM ages showed comparable performance to those using features derived from WM age affine.

FIG. 5B. Flow charts show the processes involved in calculating FIG. 5A.

FIG. 6. The macrostructural variations are present in the affine-aligned fractional anisotropy (FA) images, while minimized in the nonrigid-aligned images. Contours of regions are provided to assist in the visual inspection of brain region shapes. Yellow arrows indicate the thalamus, which appears to shrink with age in the first row (affine-aligned) but remains consistent in shape and size in the second row (nonrigid-aligned).

FIG. 7. This disclosure matches CN data points for participants who converted from CN to MCI based on sex, age, and time to event (i.e., time to first MCI diagnosis for MCI participants and time to last CN session for CN participants).

FIG. 8 shows Table 1 of this disclosure as a table of dataset characteristics used for developing and evaluating the details of this disclosure.

FIG. 9 shows Table 2 of this disclosure as a table of classifications of CN vs. AD, CN vs. MCI, and CN vs. CN* using chronological age, sex, and brain age-related features.

FIG. 10 shows Table 3 of this disclosure as a table of added value of the WM age non-linear in predicting MCI incidence.

FIG. 11 shows Table 4 of this disclosure as a life table for the survival analysis.

FIG. 12. This disclosure computes Grad-CAM attention for WM age affine and WM age nonrigid, respectively. The attention values are normalized for each image, averaged across images from the same age group, and overlaid on the MNI152 template image. WM age nonrigid is driven by more regions beyond the ventricles, in contrast to WM age affine.

FIG. 13. This schematic shows an estimated brain age vs. chronological age for participants that are (from left to right column) CN (cognitively normal), CN* (cognitively normal at present but transitioning to mild cognitive impairment), MCI (mild cognitive impairment), and AD (Alzheimer's disease). The Pearson correlation coefficients are calculated on data points within the age range of 45 to 90 years.

FIG. 14. A flow chart of a method of this disclosure is shown.

FIG. 15. A computing environment is shown that can be used to implement the methods and systems of this disclosure.

### DETAILED DESCRIPTION

This disclosure shows that by warping subject images of brains of patients in diffusion magnetic resonance imaging (dMRI), systems and methods described herein can deliberately remove the anatomical/macro-structural information, through image processing techniques. The techniques discussed below allow for building models that focus predominantly on the microstructural information in determining brain age. Compared with models that use both micro- and macro-structural information (e.g., other dMRI-based brain age estimation models), or models that use macro-structural information (e.g., T1w-based brain age estimation models), the model described herein has advantages and added value for neurodegenerative disease prediction.

Non-limiting embodiments of this disclosure include using software models and even 3D convolutional neural networks that a) focus on the microstructural features in dMRI for brain age estimation; and b) take 3D images derived from dMRI, rather than 1D feature vector(s) or other forms that require extraction of features from images beforehand, as input. To achieve 1.a, an image processing pipeline has been developed to minimize the anatomical/macrostructural information in dMRI data. The biomarker provided by software and even neural networks have complementary information to other existing brain age estimates, including the most common T1w-based brain age estimates. Overall the systems and methods of this disclosure serve as a potentially earlier biomarker for neurodegenerative disease prediction.

Estimated brain age from magnetic resonance image (MRI) and its deviation from chronological age can provide early insights into potential neurodegenerative diseases, enabling more effective clinical intervention and prevention of disease progression and onset. Diffusion MRI (dMRI), a widely used modality for brain age estimation, presents an opportunity to build an earlier biomarker for neurodegenerative disease prediction because it captures subtle microstructural changes that precede more perceptible macrostructural changes. However, the coexistence of macro- and micro-structural information in dMRI raises the question of whether current dMRI-based brain age estimation models are leveraging the intended microstructural information or if they inadvertently rely on the macrostructural information. To develop a microstructure-specific brain age, this disclosure proposed a method for brain age identification from dMRI that minimizes the model's use of macrostructural information by non-rigidly registering all images to a standard template. Imaging data from 13,398 participants across 12 datasets were used for the training and evaluation.

After quality assurance, there were a total of 13,398 participants, contributing to 18,673 imaging sessions that included both cross-sectional and longitudinal data. For every imaging session, both diffusion MRI and T1w MRI were acquired. This disclosure selected 10,647 CN participants and divided them into five folds for training and cross-validation. During training, data samples from longitudinal sessions and multiple scans were included and treated as a form of data augmentation. To avoid biasing the model toward participants who have more data samples, this disclosure normalized each sample's probability of being sampled at each iteration by the total number of samples belonging to that participant. For example, consider a training set with only two participants: A (who has two samples, dA1 and dA2), and B (who has one sample, dB1). After normalization, the probabilities of sampling dA1, dA2, and dB1 are 0.25, 0.25, and 0.5, respectively. The remaining 2,751 participants were held out as the testing set. IRB of Vanderbilt University waived ethical approval for de-identified access of the human subject data.

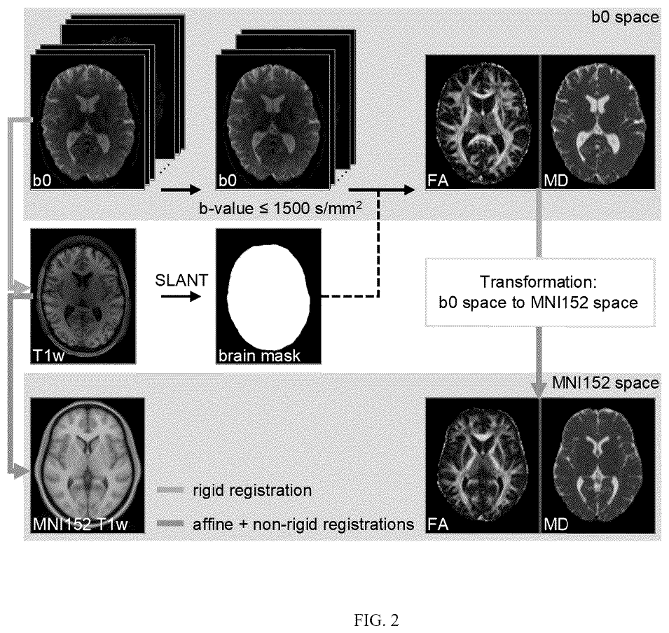

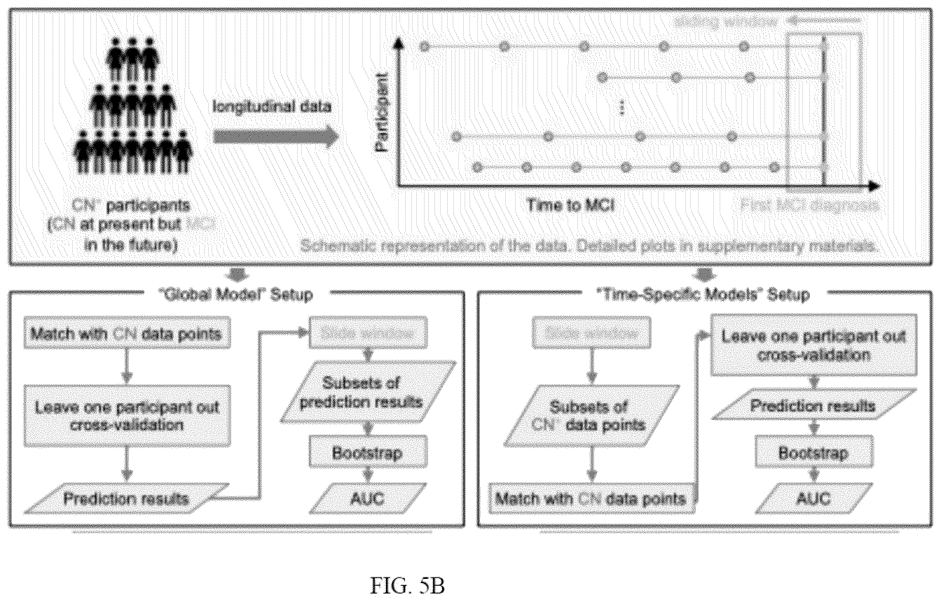

For dMRI data, this disclosure used PreQual, (L. Y. Cai et al., 2021) an end-to-end preprocessing pipeline, for denoising and to attenuate susceptibility artifacts, motion, and eddy current artifacts. This disclosure computed two diffusion tensor imaging (DTI) scalar maps, fractional anisotropy (FA) and mean diffusivity (MD), from the volumes acquired with a b-value≤1500 s/mm² and transformed them to MNI152 space (Fonov et al., 2011) (FIG. 2). There are two types of transformations: the first involves a rigid transformation (from b0 to T1w) followed by an affine transformation (from T1w to MNI152), which aligns the brain to the template while preserving macrostructural variations. The second type combines the rigid and affine transformations with a nonrigid (deformable) transformation, (Avants et al., 2008) further warping the brain to match the template and mitigate macrostructural variations (FIG. 6). For T1w images, this disclosure applied only the affine transformation to preserve macrostructural information. The registrations were performed using ANTs. (Avants et al., 2011) The nonrigid registration was done with the deformable SyN option. (Avants et al., 2008). This disclosure used SLANT-TICV, (Huo et al., 2019; Liu et al., 2022) a deep learning-based whole brain segmentation pipeline, to generate brain masks for skull-stripping the FA, MD, and T1w images.

The registered and skull-stripped images were then down-sampled and cropped to 128*152*128 with an isotropic resolution of 1.5 mm³ to reduce GPU RAM requirements. Since TSAN (Cheng et al., 2021) was originally trained on a relatively smaller dataset, (Cheng et al., 2021). This disclosure retrained it on our richer dataset to enable a fair comparison. This disclosure followed the requirements described in the paper (Cheng et al., 2021) for retraining TSAN. The images were down-sampled and cropped to 91*109*91 with an isotropic resolution of 2 mm³. For running the pretrained DeepBrainNet model, this disclosure strictly followed the preprocessing steps and software tools described in the paper. (Bashyam et al., 2020) The preprocessed images were manually checked. Table 1 reflects what remained after the quality assurance.

This disclosure compared our brain age models, trained with and without macrostructural information minimized, with an architecturally similar T1-weighted (T1w) MRI-based brain age model and two state-of-the-art T1w MRI-based brain age models that primarily use macrostructural information. This disclosure observed difference between our dMRI-based brain age and T1w MRI-based brain age across stages of neurodegeneration, with dMRI-based brain age being more advanced than T1w MRI-based brain age in participants transitioning from cognitively normal (CN) to mild cognitive impairment (MCI) (p-value=0.023), but less advanced in participants already diagnosed with Alzheimer's disease (AD) (p-value<0.001). Classifiers using T1w MRI-based brain ages generally outperform those using dMRI-based brain age in classifying CN vs. AD participants. Conversely, dMRI-based brain age yields better performance than T1w MRI-based brain ages in predicting whether a CN participant will transition to MCI in the future from 4 years before diagnosis and has added value in predicting MCI incidence in survival analysis.

Patterns of macro- and micro-structural changes associated with normal brain aging can be captured from magnetic resonance images (MRIs) by machine learning methods to construct brain age—an important imaging biomarker in the fields of neuroscience and radiology. [1] By comparing an individual's MRI-derived brain age with their chronological age, deviations from the normal ageing trajectory can be identified. A brain age that is less advanced than the chronological age may reflect good brain health and resilient ageing. [2-4] Conversely, a brain age that is more advanced than the chronological age may reflect accelerated aging, which could be indicative of neurodegenerative diseases or cognitive decline. [5-7] Early identification of at-risk individuals enables proactive management of conditions like mild cognitive impairment (MCI) or Alzheimer's disease (AD), leading to timely and targeted therapeutic strategies, which may slow disease progression. [8,9]

Specificity and sensitivity are two critical aspects of machine learning models in clinical applications. In the context of brain age estimation—where the difference between estimated brain age and chronological age (i.e., the brain age gap) can be used to classify whether an individual is developing neurodegenerative diseases—specificity relates to accurate chronological age estimation for individuals who are neither experiencing nor on a trajectory to develop neurodegenerative diseases or cognitive decline, to avoid false alarms. Sensitivity, on the other hand, relates to detecting deviations from the normal aging trajectory, as indicated by large positive brain age gaps in individuals who are either experiencing or on a trajectory to develop neurodegenerative disease or cognitive decline. Ideally, this disclosure would hope to detect such deviations well before clinical diagnosis, allowing ample time for intervention.

Considerable efforts have been made to enhance the specificity of brain age estimation models. Among these efforts, four trends stand out. First, there is a growing emphasis on using large datasets that encompass a diverse range of cohorts, characterized by variations in age, race, sex, education, and geographic location, as well as acquisitions that differ in scanner type, imaging parameters, and quality. [7,10,11] The rationale for using larger and more heterogeneous datasets is to develop models that are robust and generalizable, capable of maintaining accuracy when applied to previously unseen data. Second, the field is witnessing a paradigm shift towards the adoption of deep neural networks with sophisticated architectural designs. [7,11-14] These networks have the capacity to learn complex feature representations directly from brain images, offering an advantage over traditional machine learning models that rely on hand-crafted and preselected features. [1,5,15,16] Third, the fusion of multimodal imaging data is increasingly being used. [6,14] By combining data from different imaging modalities, models can potentially capture a wider spectrum of age-related changes. Fourth, transfer learning is being used to leverage pre-trained models on large datasets to improve performance on smaller, target datasets. [17,18] Through these efforts, the field has reported progressively lower mean absolute errors in brain age estimation for healthy individuals.

Comparatively, fewer efforts have been directed towards improving the sensitivity of brain age estimation models. [1,5,7,16] A common theme of brain age estimation methods involves the use of T1-weighted (T1w) images, which primarily capture macrostructural and intensity information. [7,12,13 T1w] images allow us to observe changes related to brain ageing, such as atrophy, [19,20] cortical thinning, ventricular enlargement, [22,23] and white matter hyperintensities. [16,24] However, T1w images lack detailed information about white matter regions, making them less sensitive to the early microstructural changes that precede noticeable macrostructural changes. [25-29] With regard to MCI and AD, emerging evidence highlights distinct white matter abnormalities, including axonal loss, 30 demyelination, [31] and microglial activation. [32] Importantly, these changes manifest up to 22 years prior to symptom onset [33,34] and have independent contributions to cognitive decline beyond that of hippocampal volume. [35] Diffusion MRI (dMRI), on the other hand, can capture white matter microstructural alterations, offering the potential to develop an earlier biomarker for neurodegenerative disease prediction. [26-28,36] Nonetheless, the presence of macrostructural information within dMRI data presents a confounding factor. It remains unclear whether current brain age estimation models based on dMRI data are leveraging the intended microstructural information or if they are inadvertently relying on macrostructural information.

In this study, this disclosure isolated the microstructural information from dMRI data for brain age estimation. Specifically, this disclosure used nonrigid (deformable) registrations to warp all brains to one standard template brain, thereby minimizing macrostructural variations across the dataset. This disclosure hypothesized that the microstructure-informed brain age will serve as an earlier biomarker for neurodegenerative diseases, offering improved predictive capabilities for conditions such as mild cognitive impairment (MCI). To serve the testing of the hypothesis, this disclosure included 12 datasets comprising a total of 13,398 participants, with longitudinal data included. For the architecture of our brain age estimation models, this disclosure used 3D residual neural network (ResNet), [37] a well-established convolutional neural network architecture in the field. To compare microstructure-informed brain age with “micro- and macro-structure mixture”—informed brain age, this disclosure trained the ResNets using dMRI-derived data with and without the macrostructural information minimized through non-rigid registrations. Additionally, to compare microstructure-informed brain age with “T1w macrostructure”-informed brain age, this disclosure also trained two separate brain age estimation models based on T1w images. One model uses the same ResNet architecture, while the other uses a state-of-the-art architecture known as TSAN13; both were trained on the same set of participants as the dMRI-based models. For a more comprehensive comparison, this disclosure also applied DeepBrainNet7, another highly regarded T1w-based brain age estimation model, to our data using pretrained model weights.

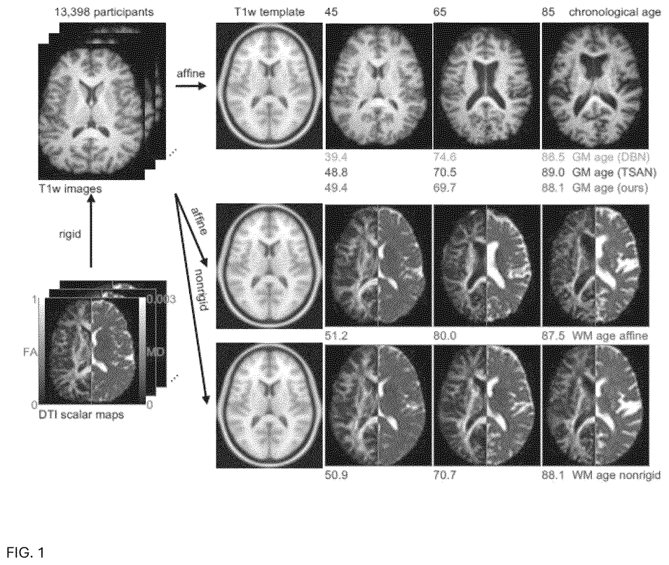

This disclosure conducted comparisons of these brain ages (FIG. 1). This disclosure examined their differences across diagnostic groups, such as cognitively normal (CN), Alzheimer's disease (AD), mild cognitive impairment (MCI), and CN participants who later transitioned to MCI. This disclosure assessed their performance in classifying participants within these groups and in predicting the likelihood of a CN participant transitioning to MCI in the future, from 0 to 9 years prior to diagnosis. Furthermore, this disclosure investigated the added value of microstructure-informed brain age on T1w-based brain ages in predicting MCI incidence in survival analysis.

### Brain Age Estimation Models

This disclosure included three types of models, each type schematized as a row in FIG. 1. The first type represents T1w MRI-based models. Since the images provide high-contrast structural information about gray matter (GM) regions, this disclosure named these models “GM age” models. Among them, this disclosure has a model (“GM age (ours)”), which uses a 3D ResNet [37] as the architecture and takes the T1w image, along with sex and race information, as input. The embedding from the convolutional layers is concatenated with the vectorized sex and race information before entering the fully connected layers to output the estimated brain age. This disclosure also included TSAN (“GM age (TSAN)”), as a comparison with a more advanced architecture that achieved state-of-the-art performance.[13] GM age (ours) and GM age (TSAN) were both trained from scratch on our skull-stripped T1w images affinely registered to the MNI152 template. Additionally, this disclosure included the pretrained DeepBrainNet [7] (“GM age (DBN)”) as another comparison method. The inference process for GM age (DBN) strictly followed the processing steps described in the paper. [7] The second type uses a similar architecture to the 3D ResNet of GM age (ours), except that it substitutes the T1w image with FA and MD images, skull-stripped and affinely registered to the MNI152 template. The FA and MD images are concatenated together as two channels before being fed into the network.

This disclosure also explores the relative timing of changes in these brain age variants and their relative explainability in the context of mild cognitive impairment. In another description of this embodiment, the method and system calculate a transformation matrix from each type of the registrations (rigid, affine, nonrigid). To get row 2, the method concatenates the transformation matrix of the rigid registration and the transformation matrix of the affine registration. Next, the method applies the concatenated transformation to the DTI scalar maps to get row 2. Similarly, the method allows for a computer to concatenate the transformation matrix of the rigid registration, the transformation matrix of the affine registration, and the transformation matrix of the nonrigid registration. The method then applies the concatenated transformation to DTI scalar maps to get row 3. The reason for the concatenation is to preserve the quality of the image as much as possible. Each time of image transformation requires interpolation. More rounds of interpolation lead to more “blurred” image. One non-limiting goal of this disclosure is to minimize the number of rounds of interpolation to one for each row.

Because the input images contain microstructural information, which shows the most variation in white matter (WM) regions, this disclosure named the model “WM age affine”. The third type, “WM age nonrigid”, uses the exact same model architecture as “WM age affine”, except that the input images are skull-stripped FA and MD images non-rigidly registered to the MNI152 template. This disclosure trained the models, except “GM age (DBN)”, using scans of individuals aged between 45 and 90 years. This range provided a sufficiently large sample of midlife to older adults, aligning with our goal of investigating age-related changes linked to MCI and AD. This disclosure excluded scans of individuals outside this range because their numbers were relatively small for both training and evaluation. This disclosure implemented two strategies to mitigate the models' bias towards middle-aged participants. First, the age of the scan is sampled uniformly during training. Scans being sampled are assigned decayed probabilities of being sampled again, ensuring all available scans can be iterated through in fewer iterations. Second, this disclosure fit bias correction parameters (slope and intercept) on the validation set (one of the five folds of the training set) after model training and apply the correction to the estimated brain ages following the steps described in detail in the paper. (Smith et al., 2019) For “GM age (DBN)”, the bias correction parameters are computed from the entire training set.

### Classification of MCI/AD Participants

To determine whether the estimated brain age by each model is indicative of neurodegeneration, this disclosure performs classification of participants by cognitive status. The features used for classification include sex, chronological age, and brain age gap (BAG), which this disclosure defined as the difference between the estimated brain age and the chronological age (Eq. 1). For participants with longitudinal sessions, this disclosure computed the change rate of the brain age gap by taking the difference between the brain age gaps from two adjacent sessions and dividing it by the interval. This disclosure generated additional features by computing interactions with chronological age and sex.

To determine whether the estimated brain age by each model is indicative of neurodegeneration, this disclosure performed classification of participants by cognitive status. For the disease status classification, this disclosure used classification models including but not limited to logistic regression, linear support vector machine, and random forest. The input to the classification model are the features used for classification, including but not limited to sex, chronological age, and brain age gap (BAG), which this disclosure defined as the difference between the estimated brain age and the chronological age (Eq. 1). For participants with longitudinal sessions, this disclosure computed the change rate of the brain age gap by taking the difference between the brain age gaps from two adjacent sessions and dividing it by the interval. This disclosure generated additional features by computing interactions with chronological age and sex.

$$BAG = Age estimated - Age chronological \qquad ( Eq . 1 )$$

This disclosure separated participants into four groups for classification. The first group consists of participants who remain CN across all available sessions. The second and third groups include participants who are diagnosed with MCI and AD, respectively. The fourth group comprises participants who are CN in the current session but will transition to MCI in future sessions, which this disclosure defined as “CN*”. This disclosure applied a greedy algorithm to obtain matched and balanced data points for group comparison and classification. Specifically, when comparing multiple (N≥2) groups of participants, this disclosure iteratively searched for data points of unused participants, one from each group, that have the same sex and the closest age, with the age difference not exceeding one year. Additionally, when matching CN and CN* data, the time to the last CN session (for the CN data point) and the time to the first MCI diagnosis (for the CN* data point) must also match, with a difference of no more than one year. This disclosure used three different machine learning classifiers for the classification: logistic regression, linear support vector machine (SVM), and random forest.

Prediction of Transition from CN to MCI

To understand the translational impact of our WM age nonrigid, this disclosure conducted a prediction experiment to determine whether brain age can predict the future transition of a CN participant to MCI. This disclosure used sliding windows (with window length of one year and stride of 0.5 year) to sample data points at various time points (T−0, T−1, . . . , T−n) before the first MCI diagnosis and assess the classifiers' ability to differentiate these data points from matched CN data points using brain age-derived features. The prediction experiment is structured into two setups, each with a distinct experimental procedure and underlying logic.

In the first setup, which is called the “global model” approach, this disclosure used the greedy algorithm to match CN data points with those transitioning to MCI (CN*). This disclosure then applied leave-one-out cross-validation, where this disclosure trained classifiers on the remaining data and test them on the left-out participant and their matched CN data points. This process is repeated for all CN* participants. Subsequently, this disclosure slid the window across the “time to MCI” axis, select the most central data point pair from each participant, and use bootstrapping to compute the mean and 95% confidence intervals of the area under the receiver operating characteristic curve (AUC) within the window.

In the second setup, which is called the “time-specific models” approach, this disclosure slid the window across the “time to MCI” axis to create subsets of data, each representing a different “time to MCI” range. For each subset, this disclosure matched CN data points using the greedy algorithm, perform leave-one-out cross-validation, and record the predicted probabilities. This disclosure then bootstrapped to compute the mean and 95% confidence intervals of the AUC for each subset. This approach utilizes multiple models, each tailored to a specific “time to MCI” range.

### Survival Analysis

To assess whether WM age nonrigid provides additional predictive value over GM ages for the incidence of MCI, this disclosure conducted survival analysis. Our cohort for this analysis includes baseline sessions from 131 CN* participants. This disclosure also incorporated baseline sessions from participants within the same datasets who remained CN until their last recorded session. The diagnosis of MCI is treated as the event of interest, with all other observations considered censored. This disclosure used Cox proportional-hazards models to evaluate the risk factors associated with MCI onset. Our analysis is structured into two scenarios: the first excluded WM age nonrigid, fitting models with chronological age, sex, and GM ages as covariates, while the second included WM age nonrigid alongside the covariates used in the first scenario.

### Statistical Analysis

For testing the null hypothesis that two related paired samples come from the same distribution, this disclosure used the Wilcoxon signed-rank test. Accuracy and AUC are reported for classification and prediction performance. The concordance index (C-index) is reported for the Cox proportional-hazards models. Bootstrapping (n=1000) is used to calculate the mean and 95% confidence intervals of these metrics. To assess fit of the Cox proportional-hazards models, this disclosure reported the Akaike information criterion (AIC) scores. This disclosure evaluated improvements in model fit using the likelihood ratio test, which compares the log-likelihoods of the nested models with and without the inclusion of WM age nonrigid. The chi-squared (c2) statistic and corresponding p-value are computed to determine the statistical significance of the improvements with the addition of WM age nonrigid. This disclosure chose an a priori threshold of pvalue<0.05 to denote statistical significance.

### Results

### Brain Age Estimation of Five Models

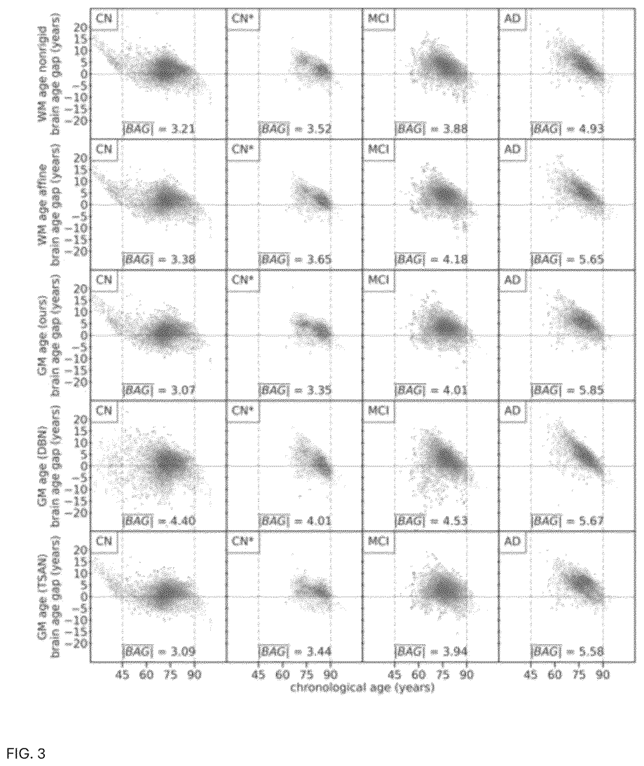

The |BAG|* (mean of absolute value of BAG) is greater in the AD group than in the MCI group, and greater in the MCI group than in the CN group (FIG. 3). This trend was also reflected in the density distribution of the BAG versus chronological age, which approached a more diagonal (sloping) shape in the AD group. In the CN* group, the |BAG|* for all models with the exception of GM age (DBN) showed an increase when compared to the CN group. For example, the |BAG|* of WM age nonrigid rose from 3.21 years in the CN group to 3.52 years in the CN* group. Among the CN participants, GM age (ours) and GM age (TSAN) achieved the lowest |BAG|* (˜3.1 years).

### Difference Between WM Age and GM Age Across Stages of Neurodegeneration

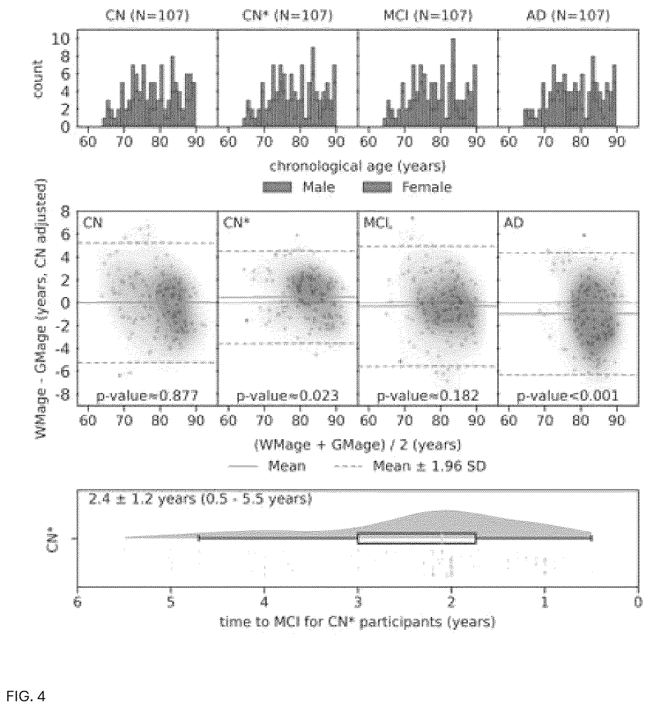

In our matched dataset, controlled for age, sex, and time-to-event, this disclosure found significant differences between WM age nonrigid and GM age (ours) among CN* participants (FIG. 4). In this group, WM age nonrigid exceeded GM age (ours) by an average of 0.48 years. A more pronounced difference was observed in participants with AD, where WM age nonrigid was, on average, 0.99 years lower than GM age (ours) (p-value<0.001). No significant differences were detected between WM age nonrigid and GM age (ours) in those who remained CN across all available sessions or those who were classified as MCI. The p-values were obtained using the Wilcoxon signed-rank test.

Classification of Cognitively Normal Vs. Current and Future Mild Cognitive Impairment and Alzheimer's Disease Participants

This disclosure conducted three classification tasks to differentiate between CN participants and those with AD, MCI, and CN participants who would later transition to MCI (CN*) (Table 2). Linear classifiers (logistic regression and linear SVM) show baseline accuracy and AUC of 0.5 with chronological age and sex, confirming that the samples are matched for these variables. As the classification task shifted from distinguishing CN vs. AD to CN vs. MCI, this disclosure observed an increase in the difficulty of classification, as reflected by decreased accuracy and AUC. In the CN vs. AD task, features derived from GM ages generally outperform those from WM age nonrigid. However, in the CN vs. MCI task, the performance gap between GM age and WM age nonrigid features narrowed. In the task of classifying CN vs. CN* participants, features derived from WM age nonrigid marginally outperform those from GM ages, although the difference is not statistically significant. Notably, combining WM age nonrigid features with GM age features consistently results in the best performance across all classification tasks.

Prediction of Transition from Cognitively Normal to Mild Cognitive Impairment from 1, . . . , n Years Pre-Diagnosis

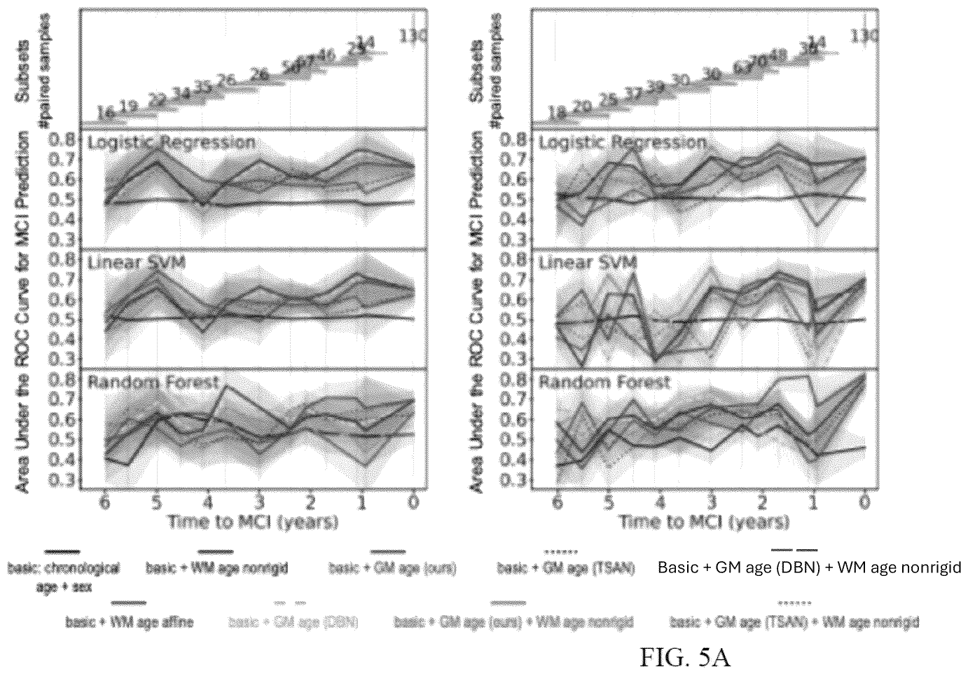

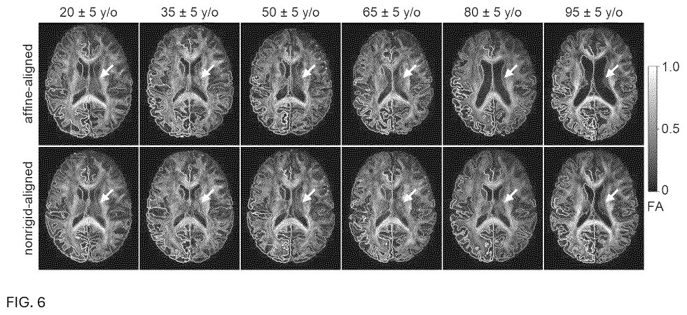

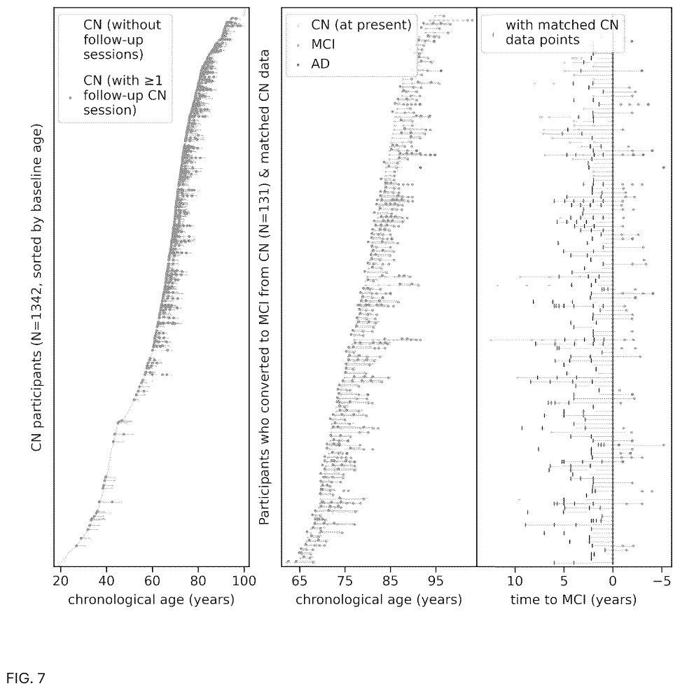

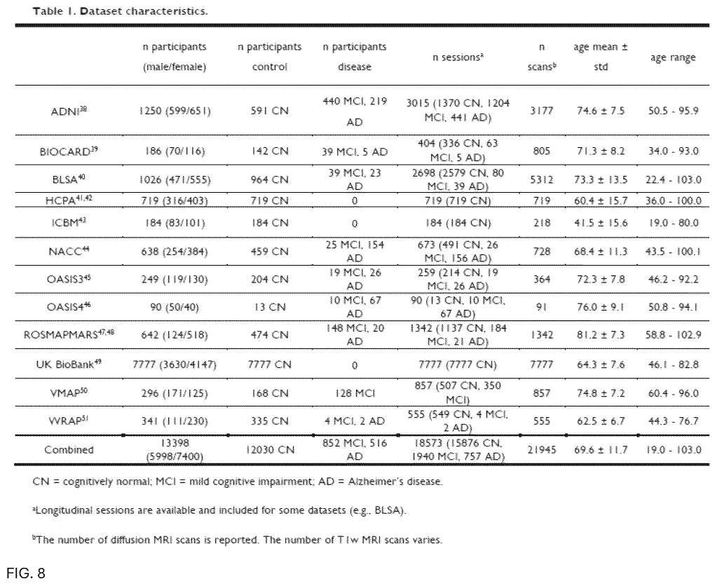

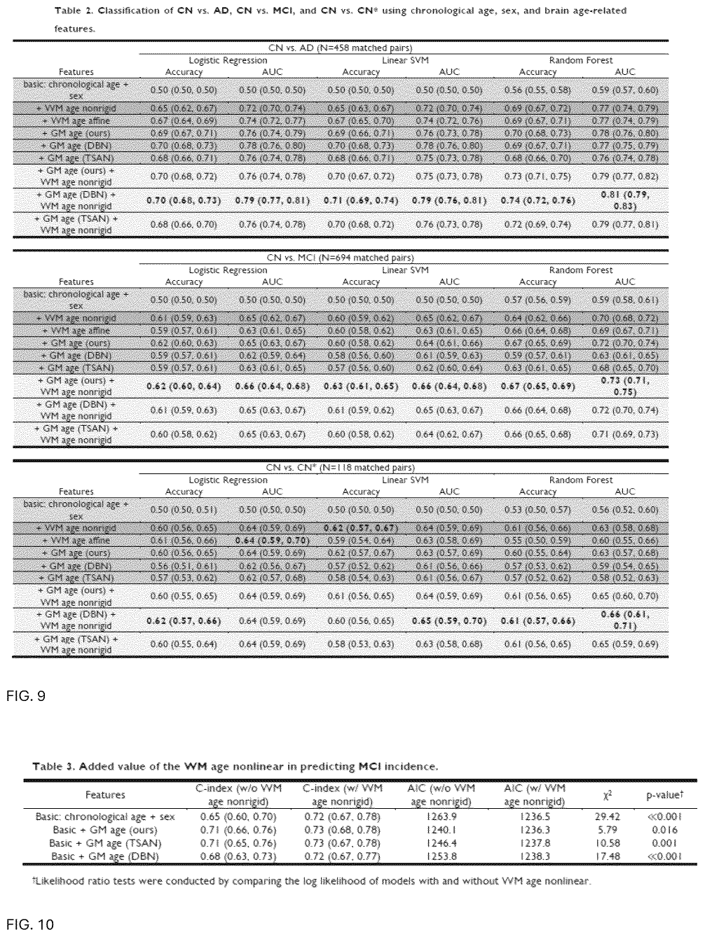

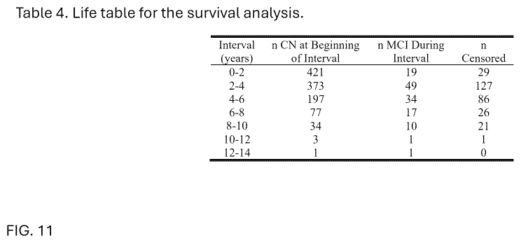

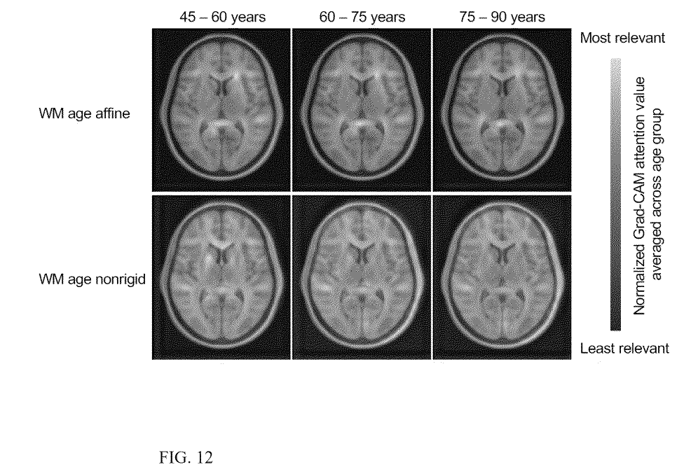

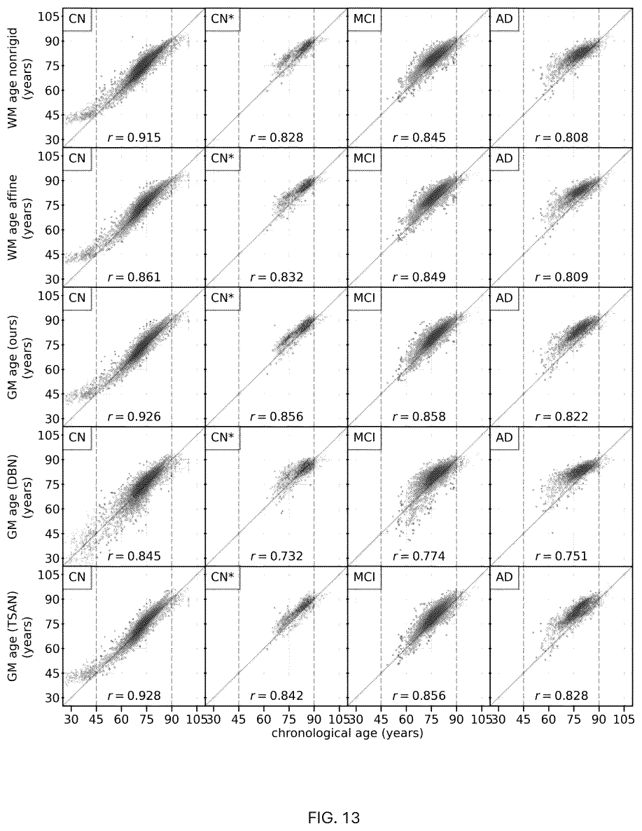

Data points of 131 participants, who had imaging data acquired from periods when they were CN and subsequent periods when they transitioned to MCI, were matched with those from CN participants (FIG. 7). At the time of MCI diagnosis (T−0), all feature combinations exhibited similar performance levels (FIG. 5). Features derived from WM ages exhibited a slight advantage across all three types of classifiers-logistic regression, linear SVM, and random forest and both the global model and time-specific models, although the differences were not statistically significant. From T−0 to T−4 (0 to 4 years before MCI diagnosis), features derived from WM age nonrigid and WM age affine, as well as their combinations with other brain age-derived features, consistently outperformed other features. Specifically, under the global model setup, random forest classifiers showed that WM age affine-derived features yielded the highest performance in the first half of this four-year period (0 to 2 years before MCI diagnosis), with an AUC of 0.7. In contrast, during the latter half (2-4 years before MCI diagnosis), WM age nonrigid-derived features achieved the best performance, with an AUC of 0.76.

At T−5 (5 years before MCI diagnosis), features derived from GM age (DBN) outperformed other features when using random forest classifiers under the global model setup, achieving an AUC of 0.78; logistic regression and linear SVM classifiers using features derived from GM ages showed comparable performance to those using features derived from WM age affine.

### Added Value of WM Age Nonrigid in Predicting Mild Cognitive Impairment Incidence

Among the 421 participants included for the survival analysis, 131 progressed to MCI, while the remaining 290 were censored. The detailed survival table can be found in the supplementary materials (Table 4). For models that included chronological age, sex, and GM ages as covariates, the addition of WM age nonrigid resulted in improvements in both the C-index and the AIC, indicating improved predictive accuracy and model fit, respectively (Table 3). Goodness-of-fit improvements resulted from the inclusion of WM age nonrigid were statistically significant (p-value<0.05).

### Discussion

GM age (ours), WM age affine, and WM age nonrigid use the same 3D ResNet architecture, with nearly identical complexity (the difference being the number of input channels). The distinct behavior of these brain ages is driven by the type of information within the images. GM age (ours) uses skull-stripped T1w images affinely registered to the MNI152 template. The T1w images capture mainly macrostructural and intensity information. WM age affine uses skull-stripped FA and MD images affine-registered to the MNI152 template. The FA and MD images contain a blend of micro- and macrostructural information. WM age nonrigid uses skull-stripped FA and MD images nonrigid-registered to the MNI152 template. The FA and MD images contain mainly microstructural information, with macrostructural information minimized. The difference in the information leads to differences in the biomarkers' properties. In diagnostic group comparisons, WM age nonrigid appears older than GM age (ours) for CN participants who will transition to MCI, suggesting that microstructural changes detectable by FA and MD are already deviating from the normal ageing trajectory, even when macrostructural changes are not yet evident in T1w images.

Conversely, for AD participants, GM age (ours) appears older than WM age nonrigid, indicating the presence of significant macrostructural changes captured by T1w images. In CN vs. AD classification tasks, classifiers using WM age affine achieved intermediate performance between those using WM age nonrigid and GM age (ours). This intermediate performance may be attributed to the macrostructural information preserved in the FA and MD images used by WM age affine. This macrostructural information enhances model performance relative to WM age nonrigid; however, due to its lower resolution (or contrast) compared to the macrostructural information in T1w images, it does not reach the performance level of GM age (ours). The pattern of WM age affine's performance falling between WM age nonrigid and GM age (ours) is consistent in MCI prediction experiments. The AUC of classifiers using WM age affine is intermediate, or its peak occurs between the peak for GM age (ours) (at 0 years) and the peak for WM age nonrigid (at 4 years prior to MCI diagnosis), as observed with the random forest in the “global model” setup and logistic regression in the “time-specific models” setup.

Our analyses demonstrate WM age nonrigid as a potential early biomarker for MCI prediction, offering added value to GM ages in forecasting MCI incidence. Starting from 4 years before MCI diagnosis, features derived from WM age nonrigid consistently outperform other brain age estimates in predicting the transition of CN participants to MCI. Likelihood ratio tests comparing nested models with and without WM age nonrigid show its contribution to improving the risk prediction of MCI incidence. In conclusion, WM age nonrigid represents a step towards improving the sensitivity of brain age estimation and can potentially benefit neurodegenerative disease prediction, prevention, and mitigation.

Current dMRI-based brain age estimation has significant overlap with structural MRI-based brain age estimation in terms of methodology, where two common approaches are (1) engineering features from the images and then using a regression model, (Gao et al., 2024; H. He et al., 2022; Wen et al., 2024) and (2) employing neural networks (typically convolutional neural networks) for representation learning on the images. (H. J. Cai et al., 2023; M. Chen et al., 2020; Gao et al., 2024; Wang et al., 2023) On the other hand, unique challenges exist in dMRI-based brain age estimation. One example is the intersite variability of dMRI data, for which researchers have proposed solutions such as transfer learning to improve model generalization. (C.-L. Chen et al., 2020)

Furthermore, this disclosure highlights two complementary directions that have emerged recently. The first is multimodal fusion, which focuses on integrating multiple imaging modalities into a single model. (H. J. Cai et al., 2023) This comprehensive approach leverages the synergistic information captured by different data types to produce an overall representation of brain aging. In contrast, the second direction is modality-specific modeling, which employs a separate model for each modality. (Wen et al., 2024) This approach generates modality-specific brain age estimates, capturing potentially distinct but interrelated neurobiological facets of aging. These modality-specific estimates offer a more nuanced view of how individual imaging measures relate to the overall aging process.

Our previous work investigated whether the unique microstructural features in dMRI could be used for brain age estimation. (Gao et al., 2024) The present study extends that work by including a larger dataset, which allows for a more comprehensive evaluation of the model. In particular, data from participants who transitioned from CN to MCI enabled us to perform classification and prediction experiments, thus examining the model's clinical value. Additionally, this disclosure compared WM age models with GM age models and provided a preliminary exploration of their relative timing in the context of neurodegeneration. When implementing the GM age models, this disclosure noticed discrepancies in the image sizes. To maintain consistency with the literature, this disclosure chose to adhere to the original settings and implementations. This disclosure acknowledges that variations in image resolution can impact the results.

In this study, this disclosure continues to use FA and MD images because they are widely used for characterizing white matter microstructural changes in brain aging. This disclosure notes, however, that other dMRI-derived measures also exist and could offer unique advantages for brain age estimation. (Roibu et al., 2023) Instead of training two separate models for males and females, this disclosure trained a single model that takes the sex label as input. This approach allows the model to learn the shared underlying features between males and females, improving data efficiency.

By selectively focusing on microstructural information for brain age estimation, this disclosure can develop a potentially more sensitive and earlier biomarker for predicting neurodegenerative diseases. Specifically, by applying nonrigid (deformable) registration to mitigate the macrostructural information in diffusion MRI data, this disclosure has derived a distinctive microstructure-informed brain age (WM age nonrigid), which holds promise as an early indicator of mild cognitive impairment. However, this disclosure acknowledges that the nonrigid registration cannot eliminate macrostructural information and can introduce artifacts that may drive the brain age estimation model. For example, Dinsdale et al. found that models trained with nonlinearly registered T1w images were driven by areas around the ventricles, and thus likely by artifacts of registration. (Dinsdale et al., 2021) To investigate whether our WM age nonrigid model uses the intended microstructural information rather than macrostructural artifacts or residuals, this disclosure used Gradient-weighted Class Activation Mapping (Grad-CAM) (Selvaraju et al., 2017) to visualize the brain regions relevant for the brain age estimation. This disclosure found that the WM age nonrigid model relies on more areas beyond the ventricles, in contrast to the WM age affine model.

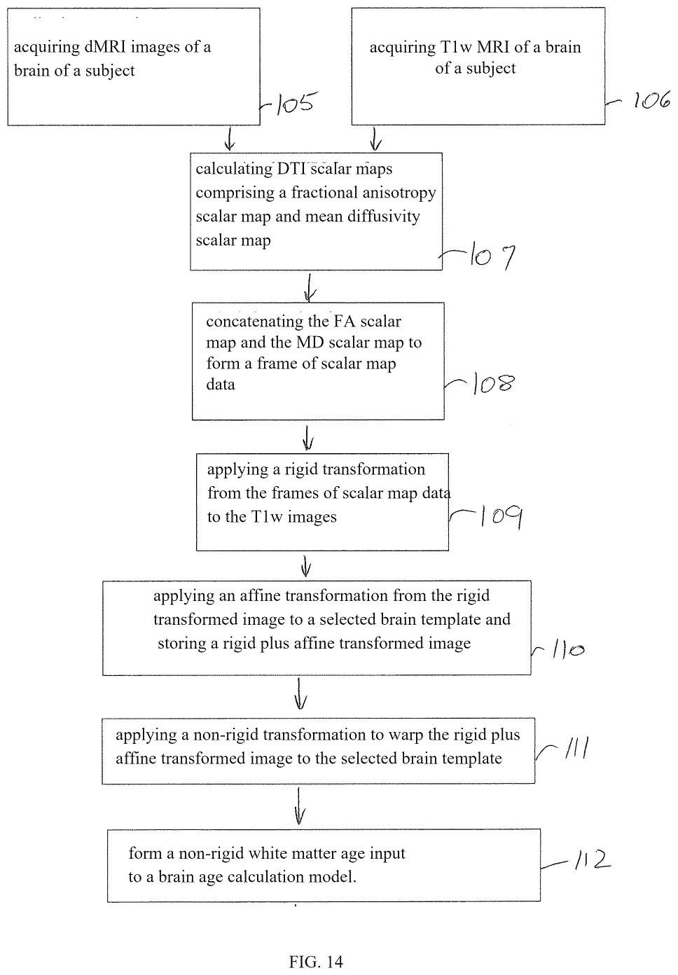

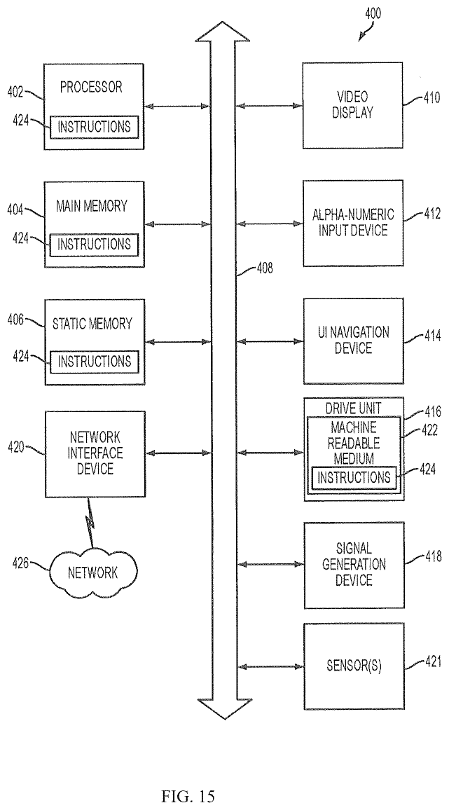

As shown in FIG. 14 and FIG. 15, embodiments of this disclosure may include, but are not limited to, a computer implemented method of preparing images of a subject's brain for calculating a brain age for the subject. The computer typically includes a processor 402 and computer-readable memory 404 storing software to implement steps as instructions 424. The steps may include, but are not limited to, acquiring diffusion magnetic resonance images (dMRI) 105 and T1 weighted magnetic resonance images (T1w MRI) 106 of the brain of the subject; calculating a pair of diffusion tensor imaging (DTI) scalar maps 107 having a fractional anisotropy (FA) scalar map and mean diffusivity (MD) scalar map; concatenating the FA scalar map and the MD scalar map 108 to form a frame of scalar map data; applying a rigid transformation 109 from the frames of scalar map data to the T1w images to store a rigid transformed image in the computer memory; applying an affine transformation 110 from the rigid transformed image to a selected brain template and storing a rigid plus affine transformed image in the computer memory; applying a non-rigid transformation to warp the rigid plus affine transformed image to the selected brain template 111 to form a non-rigid white matter age input to a brain age calculation model 112.

In non-limiting embodiments, the selected brain template is a MN152 space template.

In non-limiting embodiments, the method includes skull stripping the frame of scalar map data and the T1w images prior to applying the rigid transformation, the rigid plus affine transformation, and the non-rigid transformation.

In non-limiting embodiments, acquiring diffusion magnetic resonance images (dMRI) includes acquiring the dMRI with a b-value less than or equal to 1500 s/mm².

In non-limiting embodiments, acquiring the dMRI may include acquiring a b0 image and applying transformations in b0 space.

In non-limiting embodiments, the non-rigid transformation minimizes macrostructure features of the brain present in the frame of scalar map data of the brain.

In non-limiting embodiments, the T1w images of the brain are subject to affine registration to the selected brain template for use in a comparison evaluation for brain age estimation.

In non-limiting embodiments, the brain age calculation model utilizes a machine learning model.

In non-limiting embodiments, the brain age calculation model calculates a brain age gap defined as a difference between an estimated brain age and a chronological age of the subject.

In non-limiting embodiments, the brain age calculation model classifies the participants by cognitive status by applying features to the neural network comprising sex, chronological age, and brain age gap.

In non-limiting embodiments, classifying a plurality of subjects, for whom brain ages have been calculated, according to cognitive condition over time by sampling data points across a time frame and assessing a status of a cognitive condition according to the calculated brain age.

In non-limiting embodiments, classifying the plurality of subjects may include classifying the subjects as exhibiting cognitively normal status over a time period, mild cognitive impairment over the time period, Alzheimer's disease over the time period, and a transition from cognitively normal to mild cognitive impairment over the time period.

In non-limiting embodiments, the method includes using classifications to predict transitions from cognitively normal to mild cognitive impairment and to Alzheimer's disease according to the calculated brain age.

In non-limiting embodiments, predicting transitions may include computing change rates of brain age gaps among classes as a predictor of cognitive decline for a given classification.

In non-limiting embodiments, predicting transitions may include conducting a survival analysis to incorporate check points into the training of the neural networks.

An other embodiment may be a system for preparing images of a subject's brain for calculating a brain age for the subject and has a computer with a processor and computer readable memory connected to the processor and storing software to implement steps of this disclosure, including but not limited to, acquiring diffusion magnetic resonance images (dMRI) and T1 weighted magnetic resonance images (T1w MRI) of the brain of the subject; calculating a pair of diffusion tensor imaging (DTI) scalar maps comprising a fractional anisotropy (FA) scalar map and mean diffusivity (MD) scalar map; concatenating the FA scalar map and the MD scalar map to form a frame of scalar map data; applying a rigid transformation from the frames of scalar map data to the T1w images to store a rigid transformed image in the computer memory; applying an affine transformation from the rigid transformed image to a selected brain template and storing a rigid plus affine transformed image in the computer memory; and applying a non-rigid transformation to warp the rigid plus affine transformed image to the selected brain template to form a non-rigid white matter age input to a brain age calculation model.

Yet another embodiment may be a computer implemented method of preparing images of a subject's brain for calculating a brain age for the subject by using a computer with a processor and computer readable memory storing software to implement steps that include, but are not limited to, acquiring diffusion magnetic resonance images (dMRI) and T1 weighted magnetic resonance images (T1w MRI) of the brain of the subject; calculating at least one diffusion-derived scalar map of the brain; forming frames of scalar map data; applying a rigid transformation from the frames of scalar map data to the T1w images to store a rigid transformed image in the computer memory; applying an affine transformation from the rigid transformed image to a selected brain template and storing a rigid plus affine transformed image in the computer memory; applying a non-rigid transformation to warp the rigid plus affine transformed image to the selected brain template to form a non-rigid white matter age input to a brain age calculation model.

In additional non-limiting embodiments, calculating at least one diffusion-derived scalar map of the brain includes calculating a fractional anisotropy (FA) scalar map and mean diffusivity (MD) scalar map.

In additional non-limiting embodiments, forming frames of scalar map data may include concatenating the FA scalar map and the MD scalar map to form the scalar map data.

Data used in preparation of this article were obtained from the Alzheimer's Disease Neuroimaging Initiative (ADNI) database (adni.loni.usc.edu). As such, the investigators within the ADNI contributed to the design and implementation of ADNI and/or provided data but did not participate in analysis or writing of this report. A complete listing of ADNI investigators can be found at: http://adni.loni.usc.edu/wpcontent/and uploads/how_to_apply/ADNI_Acknowledgement_List.pdf.

### Dataset Links:

ADNI: Data used in the preparation of this article were obtained from the Alzheimer's Disease Neuroimaging Initiative (ADNI) database (adni.loni.usc.edu). The ADNI was launched in 2003 as a public-private partnership, led by Principal Investigator Michael W. Weiner, MD. The primary goal of ADNI has been to test whether serial magnetic resonance imaging (MRI), positron emission tomography (PET), other biological markers, and clinical and neuropsychological assessment can be combined to measure the progression of mild cognitive impairment (MCI) and early Alzheimer's disease (AD).
(www.adni-info.org)
BIOCARD: https://www.biocard-se.org/
BLSA: https://blsa.nih.gov/
HCPA: Data used in the preparation of this work were obtained from the Human Connectome Project (HCP) database (https://ida.loni.usc.edu/login.jsp). The HCP project (Principal Investigators: Bruce Rosen, M.D., Ph.D., Martinos Center at Massachusetts General Hospital; Arthur W. Toga, Ph.D., University of Southern California, Van J. Weeden, MD, Martinos Center at Massachusetts General Hospital) is supported by the National Institute of Dental and Craniofacial Research (NIDCR), the National Institute of Mental Health (NIMH) and the National Institute of Neurological Disorders and Stroke (NINDS). HCP is the result of efforts of co-investigators from the University of Southern California, Martinos Center for Biomedical Imaging at Massachusetts General Hospital (MGH), Washington University, and the University of Minnesota.
(https://www.humanconnectome.org/study/hcp-lifespan-aging)
ICBM: Data used in the preparation of this work were obtained from the International Consortium for Brain Mapping (ICBM) database (www.loni.usc.edu/ICBM). The ICBM project (Principal Investigator John Mazziotta, M.D., University of California, Los Angeles) is supported by the National Institute of Biomedical Imaging and BioEngineering. ICBM is the result of efforts of co-investigators from UCLA, Montreal Neurologic Institute, University of Texas at San Antonio, and the Institute of Medicine, Juelich/Heinrich Heine University—Germany.
(www.loni.usc.cdu/ICBM)
NACC: https://www.naccdata.org/
OASIS3 and OASIS4: https://sites.wustl.edu/oasisbrains/
ROSMAPMARS: https://www.rushu.rush.edu/research/departmental-research/rush-alzheimers-disease-center/rush-alzheimers-disease-center-research/epidemologic-research
UKBB: https://www.ukbiobank.ac.uk/
VMAP: https://www.vumc.org/vmac/vmap
WRAP: https://wrap.wisc.edu/

It must also be noted that, as used in the specification and the appended claims, the singular forms “a,” “an” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Although example embodiments of the present disclosure are explained in some instances in detail herein, it is to be understood that other embodiments are contemplated.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5).

Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications and publications as listed below and throughout this document are hereby incorporated by reference in their entirety herein.
1. Wen J, Zhao B, Yang Z, et al. The genetic architecture of multimodal human brain age. Nat Commun. 2024; 15(1):2604. doi:10.1038/s41467-024-46796-6.
2. Cole J H, Marioni R E, Harris S E, Deary I J. Brain age and other bodily ‘ages’: implications for neuropsychiatry. Mol Psychiatry. 2019; 24(2):266-281. doi:10.1038/s41380-018-0098-1
3. Liew S L, Schweighofer N, Cole J H, et al. Association of Brain Age, Lesion Volume, and Functional Outcome in Patients With Stroke. Neurology. 2023; 100(20):e2103-e2113. doi:10.1212/WNL.0000000000207219
4. Kolbeinsson A, Filippi S, Panagakis Y, et al. Accelerated MRI-predicted brain aging and its associations with cardiometabolic and brain disorders. Sci Rep. 2020; 10(1):19940. doi:10.1038/s41598-020-76518-z
5. Davatzikos C, Xu F, An Y, Fan Y, Resnick S M. Longitudinal progression of Alzheimer's-like patterns of atrophy in normal older adults: the SPARE-AD index. Brain. 2009; 132(8):2026-2035. doi:10.1093/brain/awp091
6. Liem F, Varoquaux G, Kynast J, et al. Predicting brain-age from multimodal imaging data captures cognitive impairment. Neuroimage. 2017; 148:179-188. doi:https://doi.org/10.1016/j.neuroimage.2016.11.005
7. Bashyam V M, Erus G, Doshi J, et al. MRI signatures of brain age and disease over the lifespan based on a deep brain network and 14 468 individuals worldwide. Brain. 2020; 143(7):2312-2324. doi:10.1093/brain/awaa160
8. Livingston G, Huntley J, Sommerlad A, et al. Dementia prevention, intervention, and care: 2020 report of the Lancet Commission. The Lancet. 2020; 396(10248):413-446. doi:10.1016/S0140-6736(20)30367-6
9. J B R, Chengjie X, S B T L, et al. Clinical and Biomarker Changes in Dominantly Inherited Alzheimer's Disease. New England Journal of Medicine. 2024; 367(9):795-804. doi:10.1056/NEJMoa1202753
10. Dufumier B, Grigis A, Victor J, Ambroise C, Frouin V, Duchesnay E. OpenBHB: a Large-Scale Multi-Site Brain MRI Data-set for Age Prediction and Debiasing. Neuroimage. 2022; 263:119637. doi:https://doi.org/10.1016/j.neuroimage.2022.119637
11. Wood D A, Kafiabadi S, Al Busaidi A, et al. Accurate brain-age models for routine clinical MRI examinations. Neuroimage. 2022; 249. doi:10.1016/j.neuroimage.2022.118871
12. Dinsdale N K, Bluemke E, Smith S M, et al. Learning patterns of the ageing brain in MRI using deep convolutional networks. Neuroimage. 2021; 224. doi:10.1016/j.neuroimage.2020.117401
13. Cheng J, Liu Z, Guan H, et al. Brain Age Estimation From MRI Using Cascade Networks With Ranking Loss. IEEE Trans Med Imaging. 2021; 40(12):3400-3412. doi:10.1109/TMI.2021.3085948
14. Cai H J, Gao Y, Liu MH. Graph Transformer Geometric Learning of Brain Networks Using Multimodal MR Images for Brain Age Estimation. IEEE Trans Med Imaging. 2023; 42(2):456-466. doi:10.1109/TMI.2022.3222093
15. Franke K, Ziegler G, Klöppel S, Gaser C. Estimating the age of healthy subjects from T1-weighted MRI scans using kernel methods: Exploring the influence of various parameters. Neuroimage. 2010; 50(3):883-892. doi:https://doi.org/10.1016/j.neuroimage.2010.01.005
16. Habes M, Erus G, Toledo J B, et al. White matter hyperintensities and imaging patterns of brain ageing in the general population. Brain. 2016; 139(4):1164-1179. doi:10.1093/brain/aww008
17. Chen C L, Hsu Y C, Yang L Y, et al. Generalization of diffusion magnetic resonance imaging-based brain age prediction model through transfer learning. Neuroimage. 2020; 217:116831. doi:https://doi.org/10.1016/j.neuroimage.2020.116831
18. Wood D A, Townend M, Guilhem E, et al. Optimising brain age estimation through transfer learning: A suite of pre-trained foundation models for improved performance and generalisability in a clinical setting. Hum Brain Mapp. 2024; 45(4):e26625. doi:https://doi.org/10.1002/hbm.26625
19. Fotenos A F, Snyder A Z, Girton L E, Morris J C, Buckner R L. Normative estimates of cross-sectional and longitudinal brain volume decline in aging and AD. Neurology. 2005; 64(6):1032-1039. doi:10.1212/01.WNL.0000154530.72969.11
20. Ridha B H, Barnes J, Bartlett J W, et al. Tracking atrophy progression in familial Alzheimer's disease: a serial MRI study. Lancet Neurol. 2006; 5(10):828-834. doi:https://doi.org/10.1016/S1474-4422(06)70550-6
21. Bakkour A, Morris J C, Dickerson B C. The cortical signature of prodromal AD. Neurology. 2009; 72(12):1048-1055. doi:10.1212/01.wn1.0000340981.97664.2f
22. Nestor S M, Rupsingh R, Borrie M, et al. Ventricular enlargement as a possible measure of Alzheimer's disease progression validated using the Alzheimer's disease neuroimaging initiative database. Brain. 2008; 131(9):2443-2454. doi:10.1093/brain/awn146
23. Ferrarini L, Palm W M, Olofsen H, van Buchem M A, Reiber J H C, Admiraal-Behloul F. Shape differences of the brain ventricles in Alzheimer's disease. Neuroimage. 2006; 32(3):1060-1069. doi:https://doi.org/10.1016/j.neuroimage.2006.05.048
24. De Groot J C, De Leeuw F E, Oudkerk M, et al. Periventricular cerebral white matter lesions predict rate of cognitive decline. Ann Neurol. 2002; 52(3):335-341. doi:https://doi.org/10.1002/ana.10294
25. Delpolyi A R, Mukherjee P, Gill K, et al. Comparing microstructural and macrostructural development of the cerebral cortex in premature newborns: Diffusion tensor imaging versus cortical gyration. Neuroimage. 2005; 27(3):579-586. doi:10.1016/J.NEUROIMAGE.2005.04.027
26. Kantarci K, Petersen R C, Boeve B F, et al. DWI predicts future progression to Alzheimer disease in amnestic mild cognitive impairment. Neurology. 2005; 64(5):902-904. doi:10.1212/01.WNL.0000153076.46126.E9
27. Müller M J, Greverus D, Dellani P R, et al. Functional implications of hippocampal volume and diffusivity in mild cognitive impairment. Neuroimage. 2005; 28(4):1033-1042. doi:https://doi.org/10.1016/j.neuroimage.2005.06.029
28. Ringman J M, O'Neill J, Geschwind D, et al. Diffusion tensor imaging in preclinical and presymptomatic carriers of familial Alzheimer's disease mutations. Brain. 2007; 130(7):1767-1776. doi:10.1093/brain/awm102
29. Weston P S J, Simpson I J A, Ryan N S, Ourselin S, Fox N C. Diffusion imaging changes in grey matter in Alzheimer's disease: a potential marker of early neurodegeneration. Alzheimers Res Ther. 2015; 7(1):47. doi:10.1186/s13195-015-0132-3
30. Sachdev P S, Zhuang L, Braidy N, Wen W. Is Alzheimer's a disease of the white matter? Curr Opin Psychiatry. 2013; 26(3). https://journals.lww.com/copsychiatry/fulltext/2013/05000/is_alzheimer_s_a_disease_of_the_white_matter_.4.aspx
31. Ihara M, Polvikoski T M, Hall R, et al. Quantification of myelin loss in frontal lobe white matter in vascular dementia, Alzheimer's disease, and dementia with Lewy bodies. Acta Neuropathol. 2010; 119(5):579-589. doi:10.1007/s00401-009-0635-8
32. Simpson J E, Ince P G, Higham C E, et al. Microglial activation in white matter lesions and nonlesional white matter of ageing brains. Neuropathol Appl Neurobiol. 2007; 33(6):670-683. doi:https://doi.org/10.1111/j.1365-2990.2007.00890.x
33. Lee S, Viqar F, Zimmerman M E, et al. White matter hyperintensities are a core feature of Alzheimer's disease: Evidence from the dominantly inherited Alzheimer network. Ann Neurol. 2016; 79(6):929-939. doi:https://doi.org/10.1002/ana.24647
34. Nasrabady S E, Rizvi B, Goldman J E, Brickman A M. White matter changes in Alzheimer's disease: a focus on myelin and oligodendrocytes. Acta Neuropathol Commun. 2018; 6(1):22. doi:10.1186/s40478-018-0515-3
35. Archer D B, Moore E E, Shashikumar N, et al. Free-water metrics in medial temporal lobe white matter tract projections relate to longitudinal cognitive decline. Neurobiol Aging. 2020; 94:15-23. doi:https://doi.org/10.1016/j.neurobiolaging.2020.05.001
36. Cox S R, Ritchie S J, Tucker-Drob E M, et al. Ageing and brain white matter structure in 3,513 UK Biobank participants. Nat Commun. 2016; 7(1):13629. doi:10.1038/ncomms13629
37. He K, Zhang X, Ren S, Sun J. Deep Residual Learning for Image Recognition.; 2016. http://image-net.org/challenges/LSVRC/2015/38.
Jack Jr. C R, Bernstein M A, Fox N C, et al. The Alzheimer's disease neuroimaging initiative (ADNI): MRI methods. Journal of Magnetic Resonance Imaging. 2008; 27(4):685-691. doi:https://doi.org/10.1002/jmri.21049
39. Sacktor N, Soldan A, Grega M, et al. The BIOCARD Index: A Summary Measure to Predict Onset of Mild Cognitive Impairment (P1.095). Neurology. 2017; 88(16 Supplement):P1.095. http://n.neurology.org/content/88/16_Supplement/P1.095.abstract
40. Shock N W. Normal Human Aging: The Baltimore Longitudinal Study of Aging. US Department of Health and Human Services, Public Health Service, National . . . ; 1984.
41. Harms M P, Somerville L H, Ances B M, et al. Extending the Human Connectome Project across ages: Imaging protocols for the Lifespan Development and Aging projects. Neuroimage. 2018; 183:972-984. doi:https://doi.org/10.1016/j.neuroimage.2018.09.060
42. Bookheimer S Y, Salat D H, Terpstra M, et al. The Lifespan Human Connectome Project in Aging: An overview. Neuroimage. 2019; 185:335-348. doi:https://doi.org/10.1016/j.neuroimage.2018.10.009
43. Kötter R, Mazziotta J, Toga A, et al. A probabilistic atlas and reference system for the human brain: International Consortium for Brain Mapping (ICBM). Philos Trans R Soc Lond B Biol Sci. 2001; 356(1412):1293-1322. doi:10.1098/rstb.2001.0915
44. Beekly D L, Ramos E M, Lee W W, et al. The National Alzheimer's Coordinating Center (NACC) Database: The Uniform Data Set. Alzheimer Dis Assoc Disord. 2007; 21(3). https://journals.lww.com/alzheimerjournal/fulltext/2007/07000/the_national_alzheimer_s_coordinating_center.9.aspx
45. LaMontagne P J, Benzinger T L S, Morris J C, et al. OASIS-3: Longitudinal Neuroimaging, Clinical, and Cognitive Dataset for Normal Aging and Alzheimer Disease. medRxiv. Published online Jan. 1, 2019: 2019.12.13.19014902. doi:10.1101/2019.12.13.19014902
46. Koenig L N, Day G S, Salter A, et al. Select Atrophied Regions in Alzheimer disease (SARA): An improved volumetric model for identifying Alzheimer disease dementia. Neuroimage Clin. 2020; 26:102248. doi:https://doi.org/10.1016/j.nicl.2020.102248
47. L Barnes L, C Shah R, T Aggarwal N, A Bennett D, A Schneider J. The Minority Aging Research Study: ongoing efforts to obtain brain donation in African Americans without dementia. Curr Alzheimer Res. 2012; 9(6):734-745.
48. Bennett D A, Buchman A S, Boyle P A, Barnes L L, Wilson R S, Schneider J A. Religious Orders Study and Rush Memory and Aging Project. Journal of Alzheimer's Disease. 2018; 64:S161-S189. doi:10.3233/JAD-179939
49. Sudlow C, Gallacher J, Allen N, et al. U K Biobank: An Open Access Resource for Identifying the Causes of a Wide Range of Complex Diseases of Middle and Old Age. PLoS Med. 2015; 12(3):e1001779-. https://doi.org/10.1371/journal.pmed.1001779
50. Jefferson A L, Gifford K A, Acosta L M Y, et al. The Vanderbilt Memory &amp; Aging Project: Study Design and Baseline Cohort Overview. Journal of Alzheimer's Disease. 2016; 52:539-559. doi:10.3233/JAD-150914
51. Johnson S C, Koscik R L, Jonaitis E M, et al. The Wisconsin Registry for Alzheimer's Prevention: A review of findings and current directions. Alzheimer's & Dementia: Diagnosis, Assessment & Disease Monitoring. 2018; 10:130-142. doi:https://doi.org/10.1016/j.dadm.2017.11.007
52. Cai L Y, Yang Q, Hansen C B, et al. PreQual: An automated pipeline for integrated preprocessing and quality assurance of diffusion weighted MRI images. Magn Reson Med. 2021; 86(1):456-470. doi:https://doi.org/10.1002/mrm.28678
53. Fonov V, Evans A C, Botteron K, Almli C R, Mckinstry R C, Collins D L. Unbiased average age-appropriate atlases for pediatric studies. Neuroimage. 2011; 54(1):313-327. doi:https://doi.org/10.1016/j.neuroimage.2010.07.033
54. Avants B B, Epstein C L, Grossman M, Gee J C. Symmetric diffeomorphic image registration with cross-correlation: Evaluating automated labeling of elderly and neurodegenerative brain. Med Image Anal. 2008; 12(1):26-41. doi:https://doi.org/10.1016/j.media.2007.06.004
55. Avants B B, Tustison N J, Song G, Cook P A, Klein A, Gee J C. A reproducible evaluation of ANTs similarity metric performance in brain image registration. Neuroimage. 2011; 54(3):2033-2044. doi:https://doi.org/10.1016/j.neuroimage.2010.09.025
56. Huo Y, Xu Z, Xiong Y, et al. 3D whole brain segmentation using spatially localized atlas network tiles. Neuroimage. 2019; 194:105-119. doi:https://doi.org/10.1016/j.neuroimage.2019.03.041
57. Liu Y, Huo Y, Dewey B, Wei Y, Lyu I, Landman B A. Generalizing deep learning brain segmentation for skull removal and intracranial measurements. Magn Reson Imaging. 2022; 88:44-52. doi:https://doi.org/10.1016/j.mri.2022.01.004
58. Smith S M, Vidaurre D, Alfaro-Almagro F, Nichols T E, Miller K L. Estimation of brain age delta from brain imaging. Neuroimage. 2019; 200:528-539. doi:https://doi.org/10.1016/j.neuroimage.2019.06.017
59. Cox D R. Regression Models and Life-Tables. Journal of the Royal Statistical Society: Series B (Methodological). 1972; 34(2):187-202. doi:https://doi.org/10.1111/j.2517-6161.1972.tb00899.x

1. A computer implemented method of preparing images of a subject's brain for calculating a brain age for the subject, the method comprising:
using a computer comprising a processor and computer readable memory storing software to implement steps comprising:
acquiring diffusion magnetic resonance images (dMRI) and T1 weighted magnetic resonance images (T1w MRI) of the brain of the subject;
calculating a pair of diffusion tensor imaging (DTI) scalar maps comprising a fractional anisotropy (FA) scalar map and mean diffusivity (MD) scalar map;
concatenating the FA scalar map and the MD scalar map to form a frame of scalar map data;
applying a rigid transformation from the frames of scalar map data to the T1w images to store a rigid transformed image in the computer memory;
applying an affine transformation from the rigid transformed image to a selected brain template and storing a rigid plus affine transformed image in the computer memory;
applying a non-rigid transformation to warp the rigid plus affine transformed image to the selected brain template to form a non-rigid white matter age input to a brain age calculation model.

2. The computer implemented method of claim 1, wherein the selected brain template is a MN152 space template.

3. The computer implemented method of claim 1, further comprising skull stripping the frame of scalar map data and the T1w images prior to applying the rigid transformation, the rigid plus affine transformation, and the non-rigid transformation.

4. The computer implemented method of claim 1, wherein acquiring diffusion magnetic resonance images (dMRI) comprises acquiring the dMRI with a b-value less than or equal to 1500 s/mm².

5. The computer implemented method of claim 4, wherein acquiring the dMRI comprises acquiring a b0 image and applying transformations in b0 space.

6. The computer implemented method of claim 1, wherein the non-rigid transformation minimizes macrostructure features of the brain present in the frame of scalar map data of the brain.

7. The computer implemented method of claim 1, wherein the T1w images of the brain are subject to affine registration to the selected brain template for use in a comparison evaluation for brain age estimation.

8. The computer implemented method of claim 1, wherein the brain age calculation model utilizes a machine learning model.

9. The computer implemented method of claim 1, wherein the brain age calculation model calculates a brain age gap defined as a difference between an estimated brain age and a chronological age of the subject.

10. The computer implemented method of claim 9, wherein the brain age calculation model classifies the participants by cognitive status by applying features to the neural network comprising sex, chronological age, and brain age gap.

11. The computer implemented method of claim 10, further comprising classifying a plurality of subjects, for whom brain ages have been calculated, according to cognitive condition over time by sampling data points across a time frame and assessing a status of a cognitive condition according to the calculated brain age.

12. The computer implemented method of claim 11, wherein classifying the plurality of subjects comprises classifying the subjects as exhibiting cognitively normal status over a time period, mild cognitive impairment over the time period, Alzheimer's disease over the time period, and a transition from cognitively normal to mild cognitive impairment over the time period.

13. The computer implemented method of claim 12, further comprising using classifications to predict transitions from cognitively normal to mild cognitive impairment and to Alzheimer's disease according to the calculated brain age.

14. The computer implemented method of claim 13, wherein predicting transitions comprises computing change rates of brain age gaps among classes as a predictor of cognitive decline for a given classification.

15. The computer implemented method of claim 14, wherein predicting transitions comprises conducting a survival analysis to incorporate check points into the training of the neural networks.

16. A system for preparing images of a subject's brain for calculating a brain age for the subject, comprises
a computer comprising a processor;
computer readable memory connected to the processor and storing software to implement steps comprising:
acquiring diffusion magnetic resonance images (dMRI) and T1 weighted magnetic resonance images (T1w MRI) of the brain of the subject;
calculating a pair of diffusion tensor imaging (DTI) scalar maps comprising a fractional anisotropy (FA) scalar map and mean diffusivity (MD) scalar map;
concatenating the FA scalar map and the MD scalar map to form a frame of scalar map data;
applying a rigid transformation from the frames of scalar map data to the T1w images to store a rigid transformed image in the computer memory;
applying an affine transformation from the rigid transformed image to a selected brain template and storing a rigid plus affine transformed image in the computer memory;
applying a non-rigid transformation to warp the rigid plus affine transformed image to the selected brain template to form a non-rigid white matter age input to a brain age calculation model.

17. A computer implemented method of preparing images of a subject's brain for calculating a brain age for the subject, the method comprising:
using a computer comprising a processor and computer readable memory storing software to implement steps comprising:
acquiring diffusion magnetic resonance images (dMRI) and T1 weighted magnetic resonance images (T1w MRI) of the brain of the subject;
calculating at least one diffusion-derived scalar map of the brain;
forming frames of scalar map data;
applying a rigid transformation from the frames of scalar map data to the T1w images to store a rigid transformed image in the computer memory;
applying an affine transformation from the rigid transformed image to a selected brain template and storing a rigid plus affine transformed image in the computer memory;
applying a non-rigid transformation to warp the rigid plus affine transformed image to the selected brain template to form a non-rigid white matter age input to a brain age calculation model.

18. The computer implemented method of claim 17, wherein calculating at least one diffusion-derived scalar map of the brain comprises calculating a fractional anisotropy (FA) scalar map and mean diffusivity (MD) scalar map.

19. The computer implemented method of claim 18, wherein forming frames of scalar map data comprises concatenating the FA scalar map and the MD scalar map to form the scalar map data.
